(19) United States
(12) Patent Application Publication
(10) Pub. No.: US 2002/0115106 A1
(43) Pub. Date: Aug. 22, 2002

(54) Methods for forming combinatorial libraries using reductive amination

(75) Inventors: Gerald W. Shipps, Stoneham, MA (US); Kollol Pal, Needham, MA (US)

Correspondence Address: HALE AND DORR, LLP, 60 STATE STREET, BOSTON, MA, 02109

(73) Assignee: NeoGenesis Pharmaceuticals, Inc., Cambridge, MA

(21) Appl. No.: 10/029,010

(22) Filed: Dec. 20, 2001

Related U.S. Application Data
(60) Provisional application No. 60/258,148, filed on Dec. 22, 2000.

(51) Int. Cl. G01N 33/53; C07C 239/00; C07C 235/00; C07C 311/00

(52) U.S. Cl. 435/7.1; 564/31; 564/80; 564/123

(57) ABSTRACT

The invention relates to methods for forming combinatorial libraries. The invention provides methods suitable for the rapid and convenient synthesis of very large combinatorial libraries of small organic molecules. In particular, the invention provides a method for forming combinatorial libraries combining amide, sulfonamide, or urea bond formation with reductive amination.

### CROSS-REFERENCE TO RELATED APPLICATIONS

This application claims the benefit of U.S. Provisional Application Serial No. 60/258,148, filed on Dec. 22, 2000.

### BACKGROUND OF THE INVENTION

1. Field of the Invention

This invention relates to methods for forming combinatorial libraries.

2. Summary of the Related Art

Pharmaceutical research depends on the identification of compounds that are capable of modulating biological reactions. One approach to finding these compounds is through rational drug design. In rational drug design, the structure of a target molecule (e.g., a protein) is determined, and potential ligands for the target are designed based on this structural information. Alternatively, compounds which are structurally related to known ligands of the target are prepared and tested for biological activity. Ideally, the structures of both the target and ligands are known, and new ligands are designed to optimize the structural complementarity of the target and ligand.

In many cases, however, lead compounds are best discovered by random screening approaches. This is especially true for those targets whose structures are unknown and for which there are no known ligands. For screening approaches to be successful, large numbers of structurally diverse compounds must be tested to increase the probability that one or more of the tested compounds will exhibit the desired activity. As screening technology has improved, a shortage in available compounds for testing has emerged as a limiting factor in drug discovery.

To address this demand, chemists have developed methodologies for the rapid synthesis of large combinatorial libraries of peptides, oligonucleotides, and small organic molecules. In combinatorial chemistry, a large number of compounds are produced, either in the same reaction vessel, or in separate vessels. The entire combinatorial library is then assayed, and active molecules are identified, isolated if necessary, and analyzed.

Due to the poor pharmacokinetic properties of many peptides and oligonucleotides, combinatorial chemistry efforts have increasingly focused on libraries of small organic molecules, as further described in Rebek et al., U.S. Pat. No. 5,877,030, and in Lenz, “Optimizing Small Molecule Drug Targets: Focus on Combinatorial Chemistry,” Decision Resources, Mar. 31, 1998, the entire contents of which are herein incorporated by reference. However, only a limited number of synthetic methods have been adapted for the preparation of large libraries of small organic molecules. There is thus a need in the art for new methods for the rapid and convenient synthesis of very large combinatorial libraries.

### BRIEF SUMMARY OF THE INVENTION

The present invention provides methods suitable for the rapid and convenient synthesis of very large combinatorial libraries of small organic molecules. In particular, the method of the invention comprises contacting a core molecule having at least two different reactive centers with a mixture of nucleophilic building blocks to form a set of compounds. Thus, according to the method of the invention, a library containing a wide variety of difunctionalized compounds can be easily prepared in a one-pot reaction.

In a first aspect, the invention provides a method of forming a combinatorial library of compounds, the method comprising reacting a plurality of core molecules with a mixture of nucleophilic building blocks in a reaction vessel to form a library of compounds, wherein each of the core molecules comprises (i) an acid halide, sulfonyl halide, isocyanate or isocyanate equivalent, or activated ester functional group; and (ii) an aldehyde or ketone functional group.

In a preferred embodiment according to this aspect of the invention, the core molecule has the formula A-B-C, wherein

B comprises from 1 to about 4 carbocyclic or heterocyclic rings, any of which rings may be optionally substituted, and wherein A and C may be attached to the same or different rings;

A is an organic moiety comprising an acid halide, sulfonyl halide, isocyanate or isocyanate equivalent, or activated ester functional group; and

C is an organic moiety comprising an aldehyde or ketone functional group.

In some preferred embodiments, A has the formula —Y¹—W, where:

W is an isocyanate or isocyanate equivalent, acid halide, or sulfonyl halide functional group, or W has the formula —C(O)—OR¹, where R¹ is selected from the group consisting of haloalkyl and aryl substituted with at least one electron withdrawing substituent, or —OR¹ is a radical formed by deprotonation of an N-hydroxyheterocycle or N-hydroxyimide;

Y¹ is absent or comprises a linking chain of from 1 to about 6 contiguous atoms independently selected from the group consisting of carbon, nitrogen, oxygen, or sulfur, wherein the carbon and nitrogen atoms may be optionally substituted and the nitrogen and sulfur atoms may be optionally oxidized, and wherein any of the contiguous atoms of the chemical linkage may form part of a ring structure; and

C has the formula —Y²—Z, where Y² is as defined above for Y¹ and Z is an aldehyde or ketone.

In a second aspect, the invention provides a combinatorial library of compounds, wherein each of said compounds is produced from the reaction of a core molecule, having (i) an acid halide, sulfonyl halide, isocyanate or isocyanate equivalent, or activated ester functional group; and (ii) an aldehyde or ketone functional group, with a mixture of nucleophilic building blocks.

In a third aspect, the invention provides a compound of the formula A-B-C, wherein

B comprises from 1 to about 4 carbocyclic or heterocyclic rings, any of which rings may be optionally substituted, and wherein A and C may be attached to the same or different rings;

A is an organic moiety comprising an acid halide, sulfonyl halide, isocyanate or isocyanate equivalent, or activated ester functional group; and

C is an organic moiety comprising an aldehyde or ketone functional group.

### BRIEF DESCRIPTION OF THE FIGURES

FIG. 1 is an overview of the creation and analysis of a combinatorial library.

FIG. 2 is a depiction of pharmacologically active core ring systems. Below each structure is the approximate number of drugs in the Compendium of Medicinal Chemistry that contain that structure.

FIG. 3 is a schematic representation of a preferred method for screening the libraries of the invention.

FIGS. 4A-4G show an exemplary set of compatible amine building blocks for use in the method of the invention. The Chemical Abstracts registry number for each building block is provided in the second column.

FIGS. 5A-5B show a valving arrangement used in ALIS screening.

### DETAILED DESCRIPTION OF THE PREFERRED EMBODIMENTS

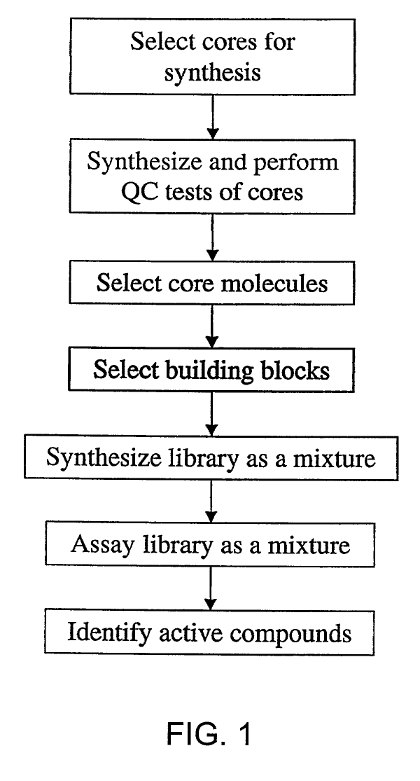

The present invention provides methods suitable for the rapid and convenient synthesis of very large combinatorial libraries of small organic molecules. In particular, the invention provides a method for forming combinatorial libraries combining amide, sulfonamide, urea, ester, carbamate, or sulfonate ester bond formation with reductive amination. A schematic representation of the method of an embodiment of the present invention is shown in FIG. 1.

The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

In a first aspect, the invention provides a method of forming a combinatorial library of compounds, the method comprising reacting a plurality of core molecules with a mixture of nucleophilic building blocks in a reaction vessel to form a library of compounds, wherein each of the core molecules comprises (i) an acid halide, sulfonyl halide, isocyanate or isocyanate equivalent, or activated ester functional group; and (ii) an aldehyde or ketone functional group. According to the invention, a large number of structurally diverse compounds are conveniently synthesized. Moreover, the library compounds produced by the present method have greater structural complexity than those prepared by other available one-pot methods for preparation of combinatorial libraries, because the core molecules used in the present method comprise at least two different reactive centers.

In particular, in preferred embodiments, the libraries produced by the present method comprise compounds having both an amine functional group and an amide, sulfonamide, urea, ester, carbamate, or sulfonate ester functional group. In preferred embodiments, at least 90%, 95%, or 99% of the library compounds each comprise an amine functional group and an amide, sulfonamide, urea, ester, carbamate, or sulfonate ester functional group.

The core molecule serves as a scaffold to which building blocks can be linked. In some embodiments, the core molecule is rigid. In the library compounds produced from such rigid core molecules, the relative position of building block moieties is fixed. In some other embodiments, the core molecule comprises a rigid portion and a non-rigid portion. In library compounds produced from these core molecules, rotation about rotatable bonds in the non-rigid portion allows for variation in the position of building blocks relative to each other and to the rigid portion of the molecule. The dynamic variability of these compounds permits conformational changes in situ, which can enhance the probability of binding to a given target.

In some embodiments, the core molecule is a dicore, and has just one acid halide, sulfonyl halide, isocyanate or isocyanate equivalent, or activated ester functional group and one aldehyde or ketone functional group. In some other embodiments, the core molecule is a tricore, and has three reactive centers, preferably one aldehyde or ketone functional group and two acid halide, sulfonyl halide, isocyanate or isocyanate equivalent, or activated ester functional groups. In yet other embodiments, the core molecule is a tetracore having four reactive centers, preferably one aldehyde or ketone functional group and three acid halide, sulfonyl halide, isocyanate or isocyanate equivalent, or activated ester functional groups.

In a preferred embodiment according to this aspect of the invention, the core molecule has the formula A-B-C, wherein

B comprises from 1 to about 4 carbocyclic or heterocyclic rings, any of which rings may be optionally substituted, and wherein A and C may be attached to the same or different rings;

A is an organic moiety comprising an acid halide, sulfonyl halide, isocyanate or isocyanate equivalent, or activated ester functional group; and

C is an organic moiety comprising an aldehyde or ketone functional group.

In some preferred embodiments, B comprises at least one aromatic ring. In some preferred embodiments, B comprises at least one heterocyclic ring. In some preferred embodiments, the heterocyclic ring is aromatic. In some preferred embodiments, B comprises a fused bicyclic or tricyclic ring system. In some other preferred embodiments, B comprises two rings connected by a covalent bond.

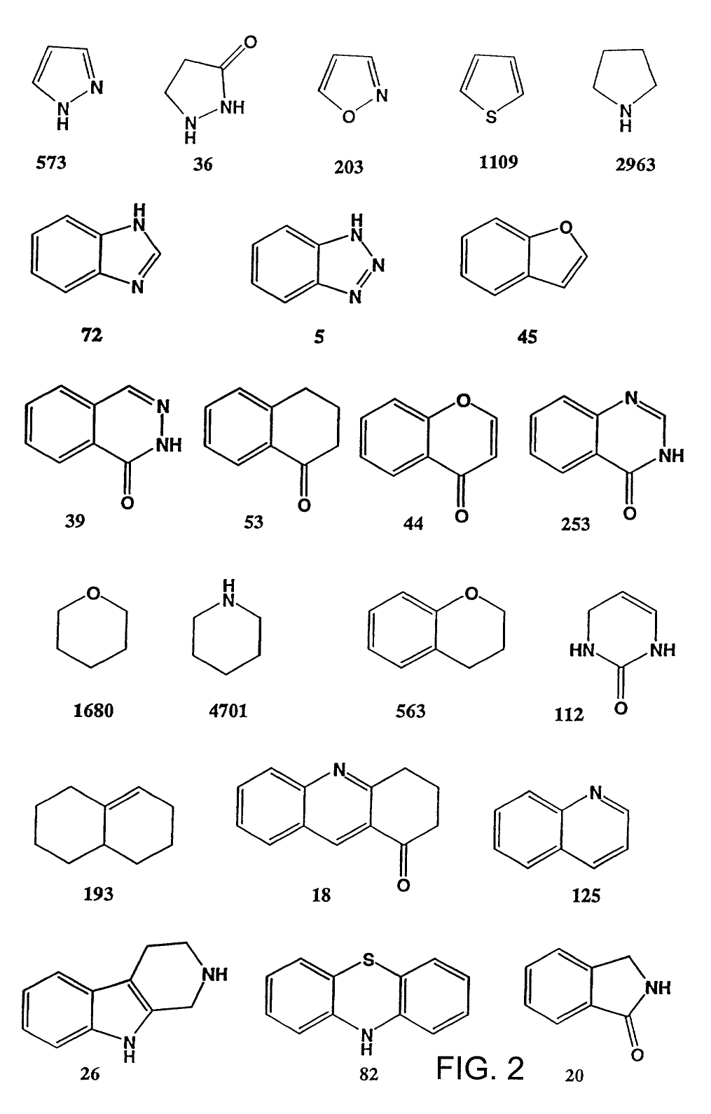

In certain preferred embodiments, B comprises a ring system with known pharmacological activity. Non-limiting examples of such pharmacologically active ring systems contemplated within the scope of the invention are depicted in FIG. 2. Below each structure is the approximate number of drugs in the Compendium of Medicinal Chemistry (CMC-3d Release 94.1; MDL Information Systems, Inc., San Leandro, Calif.) that contain that structure. The present invention provides methods for rapidly synthesizing large numbers of structural analogs of these drugs. The libraries produced by the method of the invention and the component library compounds are thus also expected to exhibit biological activity.

It will be recognized by one skilled in the art that the groups A and C may be attached to B, including the ring systems shown in FIG. 2, at any ring positions that result in a stable compound. Furthermore, A and C may be attached to the same or to different rings. The choice of position of attachment for the groups A and C will be guided by such factors as ease of synthesis and desired proximity of the building block moieties in the resultant library compounds. If A and C are attached at adjacent positions of a rigid ring, then the building block moieties will be positioned relatively close to each other in the library compounds. If A and C are attached at opposite positions of a rigid ring, then the building block moieties will be held relatively far apart in the library compounds.

In some preferred embodiments, A has the formula —Y¹—W, where:

W is an isocyanate or isocyanate equivalent, acid halide, or sulfonyl halide functional group, or W has the formula —C(O)—OR¹, where R¹ is selected from the group consisting of haloalkyl and aryl substituted with at least one electron withdrawing substituent, or —OR¹ is a radical formed by deprotonation of an N-hydroxyheterocycle or N-hydroxyimide;

Y¹ is absent or comprises a linking chain of from 1 to about 6 contiguous atoms independently selected from the group consisting of carbon, nitrogen, oxygen, or sulfur, wherein the carbon and nitrogen atoms may be optionally substituted and the nitrogen and sulfur atoms may be optionally oxidized, and wherein any of the contiguous atoms of the chemical linkage may form part of a ring structure.

Preferably, the acid halide or sulfonyl halide is an acid chloride or sulfonyl chloride.

In some preferred embodiments, W is —C(O)—OR¹, where R¹ is selected from the group consisting of haloalkyl and aryl substituted with at least one electron withdrawing substituent. In some other preferred embodiments, —OR¹ is a radical formed by deprotonation of an N-hydroxyimide or N-hydroxyheterocycle. Preferred N-hydroxyimides include, without limitation, N-hydroxysuccinimide and N-hydroxyphthalimide. A nonlimiting example of a preferred N-hydroxyheterocycle is N-hydroxybenzotriazole.

In embodiments wherein R¹ is haloalkyl, it is preferably perhaloalkyl, more preferably perfluoroalkyl, including, without limitation, trifluoromethyl, pentafluoroethyl, or heptafluoropropyl.

When R¹ is aryl, it is substituted with an electron withdrawing substituent preferably selected from the group consisting of chloro, fluoro, and nitro. More preferably, R¹ is selected from the group consisting of pentafluorophenyl, dinitrophenyl, nitrophenyl, difluorophenyl, fluorophenyl, trifluorophenyl, chlorophenyl, dichlorophenyl, chloronitrophenyl, and tetrafluoronitrophenyl.

In certain preferred embodiments, Y¹ is absent, and the acid halide, sulfonyl halide, isocyanate or isocyanate equivalent, or activated ester functional group is covalently attached directly to a ring in B. In certain other preferred embodiments, Y¹ is C₁-C₆ alkylene or C₂-C₆ alkenylene, preferably C₁-C₄ alkylene or C₂-C₄ alkenylene, any of which groups may be optionally substituted. In some preferred embodiments, the Y¹ linking chain comprises an ester, amide or sulfonamide linkage. In some other preferred embodiments, the Y¹ linking chain comprises an ether linkage.

In some preferred embodiments, C has the formula —Y²—Z, where Y² is as defined above for Y¹ and Z is an aldehyde or ketone. In certain preferred embodiments, Y² is absent, and the aldehyde or ketone functional group is covalently attached directly to a ring in B. In certain other preferred embodiments, Y² comprises a ring, and the aldehyde or ketone functional group is attached to the ring. In yet other preferred embodiments, Y² is C₁-C₆ alkylene or C₂-C₆ alkenylene, preferably C₁-C₄ alkylene or C₂-C₄ alkenylene, any of which groups may be optionally substituted. In yet other preferred embodiments, the Y² linking chain comprises an ester, amide or sulfonamide linkage. In still yet other preferred embodiments, the Y² linking chain comprises an ether linkage.

Preferably, C comprises an aldehyde functional group. More preferably, C has the formula —Y²—C(O)—H, where Y² is as defined above.

In some preferred embodiments, A comprises two W groups or C comprises an aldehyde or ketone and a W group. Preferably, A has the formula —Y¹—W, as defined above, where Y¹ comprises a W group attached to the linking chain, or C has the formula —Y²—Z, as defined above, where Y² comprises a W group attached to the linking chain.

Nonlimiting examples of useful core molecules include the following:

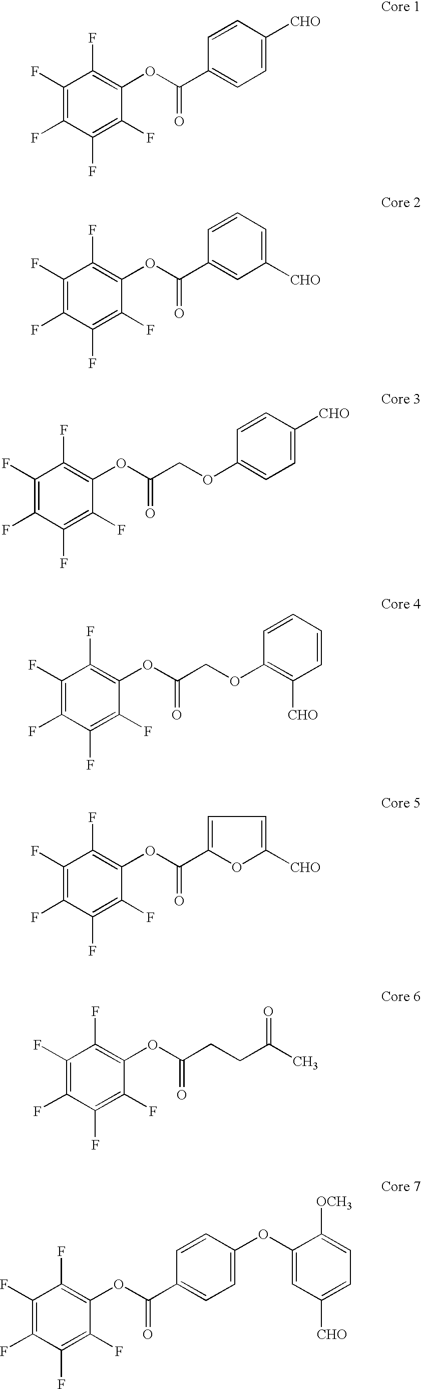

The core molecules are preferably prepared by introducing an acid halide, sulfonyl halide, isocyanate or isocyanate equivalent, or activated ester functional group into a molecule containing an aldehyde or ketone functional group. One of skill in the art is aware of a variety of synthetic approaches for the introduction of these functional groups into organic molecules. Larock, Comprehensive Organic Transformations, 2nd Edition; John Wiley-VCH: New York, 1999, provides a comprehensive description of such functional group transformations.

Synthetic routes for the preparation of a variety of core molecules within the scope of the present invention are illustrated in Equations 1-5.

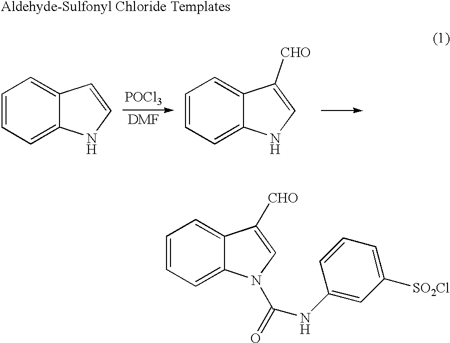

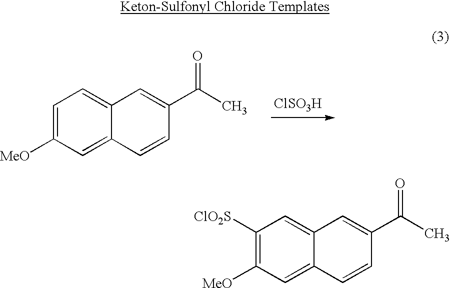

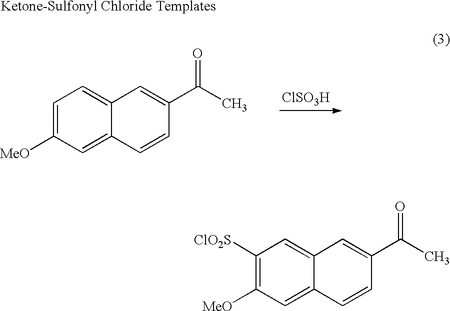

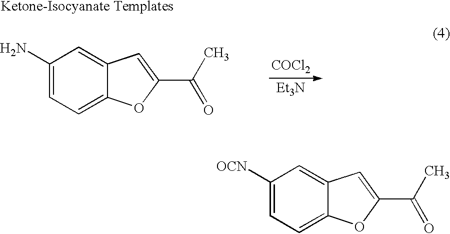

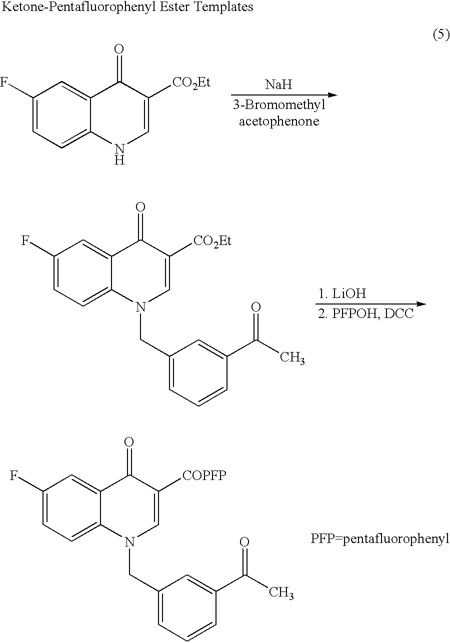

One of skill in the art would recognize that a number of reaction conditions may be utilized to carry out the transformations illustrated in these reaction sequences and that other routes may also be suitable for synthesis of the same molecules. Furthermore, one of skill in the art would recognize that other core molecules within the scope of the present invention may be synthesized by adaptation of the illustrated routes or known procedures. The synthesis of core molecules is further described in the Examples.

The core molecules are contacted with a mixture of nucleophilic building blocks to form a library of compounds. Building blocks have at least one functional group that reacts with the reactive centers of the cores to form the combinatorial library molecules. The building blocks can include more than one functional group, e.g., a first functional group for reacting with a core and a second functional group that can be protected with a protecting group. The protecting group can be removed under conditions different than those for the reaction of the core with the building blocks. The protecting groups are reversibly attached to the functional groups and can be removed from the functional groups and/or library molecules. Standard functional group protecting groups are well known to those of skill in the art and may be found described in Greene and Wuts, Protective Groups in Organic Synthesis, 2^{nd}Edition, John Wiley & Sons: New York, 1991, and in Kocienski, Protecting Groups, Georg Thieme Verlag: New York, 1994.

In some preferred embodiments, the nucleophilic building blocks are amines, preferably primary or secondary amines. The amines may be arylamines, including anilines; heteroarylamines; or alkylamines. Other nonlimiting examples of nucleophilic building blocks suitable for use in the present invention include thiols and alcohols, either of which may be either alkyl, aryl, or heteroaryl.

In some embodiments, the building blocks are preferably chosen so that each building block can react with each of the reactive centers on the core molecule. For example, amines can react with activated ester groups, acid halides, isocyanates or isocyanate equivalents, or sulfonyl halides, and with aldehydes or ketones. In some other embodiments, different building block sets are reacted with the acid halide, sulfonyl halide, isocyanate or isocyanate equivalent, or activated ester reactive centers and with the aldehyde or ketone reactive centers. For example, amine building blocks may be used for reaction with the aldehyde or ketone reactive centers, while the building blocks that are used for reaction with the acid halide, sulfonyl halide, isocyanate or isocyanate equivalent, or activated ester reactive centers may comprise alcohol, amine, or thiol functional groups.

In one embodiment, building blocks are chosen to provide for maximum diversity of the library that results when the building blocks and the cores are combined. This is preferably done by first grouping building blocks into sets of building blocks having similar molecular shape and functionality. For example, one such group of building blocks may be characterized in that all member building blocks have a non-aromatic ring and a tertiary amine. A set of building blocks to be reacted with a core molecule is then chosen such that the set comprises at least one building block from each group.

In another embodiment, building blocks are chosen to provide for a high degree of similarity between the library compounds. The method according to this embodiment of the invention is particularly useful when optimizing for a particular activity once a lead compound has been identified.

The criteria used to select the individual building blocks for inclusion into sets of building blocks for reaction with the core molecules are further elaborated below.

In one embodiment, the building blocks are selected such that each of the compounds in the resulting library has a different molecular weight than all of the other compounds in the library. The library produced according to this embodiment is referred to as a “mass-coded library”. The percentage of compounds having a molecular weight that is the same as the molecular weight of another compound in the library is referred to as the “mass redundancy” of the library.

The selection of building blocks is done, for example, as described in Nash et al., WO 99/35109, which is hereby incorporated by reference in its entirety. Briefly, a set of building blocks that can react with a core molecule having n reactive centers is selected. The set is preferably selected such that at least about 80%, 85%, 90% or 95% of the possible combinations of n building blocks derived from the set have an exact molecular mass sum that, at a resolution of 2 decimal places, is distinct from the molecular mass sum of any other combination of n building blocks derived from the set. The molecular mass sum of a combination of building block moieties is the sum of the masses of each building block moiety within the combination. Since the core is the same for each compound in the library, the differences in the molecular masses of the library compounds depend on the building blocks, and not the core. For the purposes of the invention, two molecular masses are distinct if they can be distinguished by mass spectrometry or high resolution mass spectrometry. For example, molecular masses that differ by at least 0.05 atomic mass units (AMU) can be distinguished by high resolution mass spectrometry.

In another embodiment, nucleophilic building blocks are first subjected to a test protocol to determine suitability for library synthesis. Potential building blocks are first contacted with a test core molecule having only one reactive functional group. To pass the first stage of the test protocol, a potential building block must react with the test core to give a product with a mass spectrum consistent with the predicted product, and the product preferably must be >75% pure, more preferably >80% pure, and still more preferably >85% pure as determined by a standard analytical method such as high performance liquid chromatography (HPLC). Structural and functional analogues of all potential building blocks are preferably subjected to the test protocol.

After confirming that representative individual nucleophilic building blocks each react with the test core to form a pure compound, other experiments are conducted to show that a set of representative building blocks reacts with a core molecule having at least two reactive centers to form a high percentage of the possible combinations. In other words, building blocks must be competitive in reactivity in order to be included in the final set of compatible amines. Preferably, when a set of amine building blocks is contacted with a di- or tricore, >60% (dicore) or >70% (tricore) of all possible combinations are easily detected by mass spectrometry (MS) or liquid chromatography/mass spectrometry (LC/MS).

By this procedure, it is possible to maximize the number of distinct library compounds that will be produced by contacting the core molecule with the set of amine building blocks. However, it is not possible by this procedure to determine whether all positional isomers are represented in the library. Positional isomers are library compounds that each have the same combination of building blocks, but where the building blocks are present at different positions of the core molecule. Because positional isomers have identical masses, the mass spectrometer is not able to differentiate between them. However, they can be distinguished using other analytical tools, including MS/MS analysis.

In some particularly preferred embodiments, potential building blocks are first subjected to the test protocol described above to select a set of compatible building blocks. From the set of compatible building blocks, a final set of building blocks is then selected such that each of the compounds in the resulting library has a different molecular weight than all of the other compounds in the library. Preferably, the set is selected such that at least about 80%, 85%, 90% or 95% of the possible combinations of n building blocks derived from the set have an exact molecular mass sum that, at a resolution of 2 decimal places, is distinct from the molecular mass sum of any other combination of n building blocks derived from the set.

To form a combinatorial library of compounds, a plurality of core molecules, as described above, is contacted with a mixture of nucleophilic building blocks, as described above. Preferably, the core molecules are contacted with the mixture of nucleophilic building blocks in a single reaction vessel. In some embodiments, a plurality of identical core molecules is contacted with the mixture of nucleophilic building blocks. In some other preferred embodiments, the plurality of core molecules comprises two or more different core molecules. When multiple different core molecules are contacted with the mixture of nucleophilic building blocks in a single reaction vessel, the core molecules must be compatible. That is, each core molecule must be capable of reacting with each nucleophilic building block, with essentially no reaction occurring between core molecules.

In some preferred embodiments, the reaction vessel also contains a solvent, preferably a solvent in which the core and building blocks are soluble. Nonlimiting examples of useful solvents include tetrahydrofuran (THF), dichloromethane (DCM), toluene, isopropanol, dimethylsulfoxide (DMSO), dimethyl formamide (DMF), dimethyl ether (DME), methanol, ethanol, and mixtures of these solvents.

In some preferred embodiments, base is added to the reaction mixture to neutralize the acidic leaving group and facilitate the addition of building blocks to the reactive centers on the cores. In addition, base may be required in some cases to liberate the free building block from a salt form. Useful bases include tertiary or aromatic amine bases, including, without limitation, triethylamine (Et₃N, TEA), diisopropylethylamine (DIEA), pyridine, and 4-(dimethylamino)pyridine (DMAP). Basic resins may also be used. Nonlimiting examples of such resins include Amberlyst 21 (available from Aldrich Chemical Co., Milwaukee, Wis.), Amberlyst 27 (available from Sigma Corp., St. Louis, Mo.), Dowex® 66 (available from Aldrich), Dowex® 1X8-50 (available from Aldrich), Dowex® 1X2-100(available from Aldrich), Amberlite® IRA-67 (available from Aldrich), Amberlite® IRA-900 (available from Aldrich), and Amberjet™ 4200 (available from Aldrich).

The building blocks are contacted with the core molecules under conditions and for a time sufficient for the reaction of each reactive center of the core molecule with a building block. In certain preferred embodiments, the same building block set is used for reactions at acid halide, sulfonyl halide, isocyanate or isocyanate equivalent, or activated ester sites and for reactions at aldehyde or ketone sites. In this embodiment, a plurality of core molecules is contacted with a mixture of amine building blocks to form a mixture of intermediate compounds. The amine building blocks react with acid halide or activated ester reactive centers to form amide groups; with isocyanate or isocyanate equivalent reactive centers to form urea groups; with sulfonyl halide reactive centers to form sulfonamide groups; and with aldehyde or ketone reactive centers to form imine groups.

Once reaction of the building blocks with the reactive centers is complete, a reducing agent is added to reduce the imine groups and form a combinatorial library of compounds. Preferably, the reducing agent selectively reduces imine groups without affecting amide, sulfonamide, urea, ester, carbamate, or sulfonate ester groups, and without affecting other functional groups that may be present in the core or building block portions of the molecules. Suitable reducing agents include, without limitation, sodium cyanoborohydride, sodium borohydride, sodium triacetoxyborohydride, borane pyridine, zinc borohydride, silica gel-zinc borohydride, and zinc/acetic acid. Reduction may also be accomplished by catalytic hydrogenation.

In certain preferred embodiments, the reducing agent is attached to a solid support to facilitate separation of excess reducing agent from the library compounds. Non-limiting examples of polymer-supported borohydride reagents include borohydride on Amberlite® IRA-400 (available from Aldrich), borohydride on Amberlyst® A-26 (available from Aldrich), and cyanoborohydride on Amberlyst® A-26 (available from Alfa Aesar, Ward Hill, Mass.) In some preferred embodiments, a solid support having attached aldehyde or isocyanate functional groups may be added to scavenge unreacted amine building blocks. Separation of the library compounds from excess reagents is preferably accomplished in one step by filtration. If necessary, further purification may be accomplished by a chromatographic step, preferably under reverse phase conditions.

A representative synthesis, using a single amine building block, is shown below.

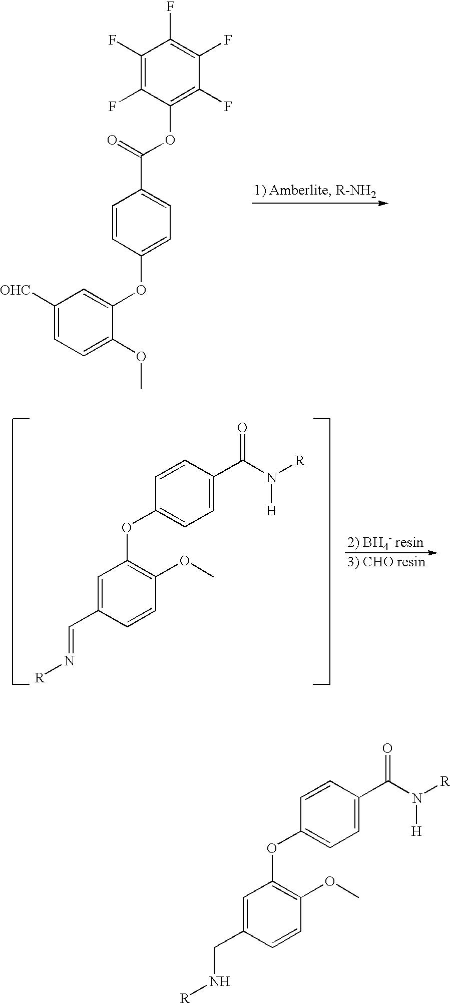

In certain other preferred embodiments, different building block sets are used for reactions at acid halide, sulfonyl halide, isocyanate or isocyanate equivalent, or activated ester sites and for reactions at aldehyde or ketone sites. In this embodiment, the acid halide, sulfonyl halide, isocyanate or isocyanate equivalent, or activated ester sites and for reactions at aldehyde or ketone reactive centers on the core molecules are each contacted with and can each react with a mixture of building blocks, wherein each building block comprises a functional group independently selected from the group consisting of alcohols, thiols, and amines. Preferably, a base, preferably a tertiary amine or aromatic amine base or a basic resin, is added to the reaction mixture to accelerate the reaction of the building blocks with acid halide, sulfonyl halide, isocyanate or isocyanate equivalent, or activated ester reactive centers on the core molecules. The aldehyde or ketone reactive centers on the core molecules are each contacted with and can each react with a mixture of amine building blocks.

In some embodiments, the two contacting steps are performed at the same time, in which case amine functional groups present in the building block mixture may react with both acid halide, sulfonyl halide, isocyanate or isocyanate equivalent, or activated ester reactive centers and with aldehyde or ketone reactive centers. In some other embodiments, the contacting steps are performed sequentially. For example, the acid halide, sulfonyl halide, isocyanate or isocyanate equivalent, or activated ester reactive centers are first contacted with a mixture of alcohol or thiol building blocks to form a first mixture of intermediate compounds. After reaction of the acid halide, sulfonyl halide, isocyanate or isocyanate equivalent, or activated ester reactive centers is substantially complete, the aldehyde or ketone reactive centers are then contacted with a mixture of amine building blocks to form a second mixture of intermediate compounds comprising imine groups. The second mixture of intermediate compounds is then contacted with a reducing agent to produce a combinatorial library of compounds.

In preferred embodiments, the amounts of core molecules and building blocks used in the coupling reaction are selected so that the molar ratio of building block functional groups to core reactive centers is equal to, or slightly greater than, one. This selection criterion enhances the likelihood that each core reactive center will be linked to a building block and that each building block (regardless of whether it contains a strong or a weak nucleophilic functional group) will react with a core reactive center. For example, 10 moles of a core having 4 reactive centers is optimally contacted with a total of 40 moles, or slightly more than 40 moles, of a plurality of amine building blocks.

If the molar ratio of building block functional groups to reactive centers is significantly greater than 1.0, the diversity of the combinatorial library may be limited, because only the most reactive centers will react with the reactive centers. If the molar ratio of building block functional groups to reactive centers is less than 1.0, the combinatorial library compounds may include unreacted reactive centers. Preferably, a ratio of slightly greater than one (e.g., 1.1 building block functional groups per core reactive site) is employed to maximize total reactivity with the cores. Because aldehydes and ketones react at a slower rate than do acid halides or activated esters, a portionwise addition of amines may preferably be implemented so that the effective ratio of 1:1 is maintained throughout the reaction.

Using the techniques described above, libraries can be generated in which each library compound is present in the library at approximately the same concentration (preferably within 100%, more preferably within 20%, and still more preferably within 10%) as any other library compound. In determining the concentration of each library compound, however, the apparent concentration must be normalized by a factor that takes into account the redundancy of positional isomers. As stated above, the mass spectrometer cannot distinguish between positional isomers, and all positional isomers must be considered to be the same compound. For example, for a library made with a core having three reactive centers and building blocks A and B, the compounds AAB, ABA, and BAA are indistinguishable. Therefore, assuming a statistical distribution of building blocks, the apparent concentration of an AAB molecule will be three times greater than the concentration of an AAA molecule.

In a preferred embodiment, the library contains at least about 100 different library compounds, more preferably at least 500, 1000, 5000, or more different library compounds. Preferably, each library compound is present in the library at the same approximate concentration as any other member of the library.

In a second aspect, the invention provides a combinatorial library of compounds, wherein each of said compounds is produced from the reaction of a core molecule, having (i) an acid halide, sulfonyl halide, isocyanate or isocyanate equivalent, or activated ester functional group; and (ii) an aldehyde or ketone functional group, with a mixture of nucleophilic building blocks.

Preferred libraries produced by the present method comprise compounds having both amine functional groups and amide, sulfonamide, urea, ester, carbamate, or sulfonate ester functional groups. In preferred embodiments, at least 90%, 95%, or 99% of the library compounds each comprise an amine functional group and an amide, sulfonamide, urea, ester, carbamate, or sulfonate ester functional group. If the core molecule has more than one acid halide, sulfonyl halide, isocyanate or isocyanate equivalent, or activated ester functional group, then a library compound may have more than one amide, sulfonamide, urea, ester, carbamate, or sulfonate ester functional group. If the core molecule has more than one aldehyde or ketone functional group, then a library compound may have more than one amine functional group.

The amine and amide, sulfonamide, urea, ester, carbamate, or sulfonate ester functional groups have electronic properties that make them useful for binding to biomolecules, including, without limitation, enzyme active sites, receptor binding sites, and other protein pockets. For example, the amine and amide, sulfonamide, urea, ester, carbamate, or sulfonate ester groups can participate in hydrogen bonding with protein residues. The combination of a secondary amine functional group with an amide, urea, or sulfonamide functional group is found in a number of compounds of known pharmacological activity. Shown in Table 1 are the number of compounds with the indicated functional group combinations that are in the MDL Drug Data Report (MDDR), and in the Compendium of Medicinal Chemistry (CMC-3d Release 94.1; MDL Information Systems, Inc., San Leandro, Calif.)

In preferred embodiments, the libraries produced by the method of the present invention possess biological activity selected from the group consisting of protease inhibitory activity, kinase inhibitory activity, antibacterial activity, antifungal activity, and anticancer activity.

In some preferred embodiments according to this aspect of the invention, the core molecule has the formula A-B-C, wherein

B comprises from 1 to about 4 carbocyclic or heterocyclic rings, any of which rings may be optionally substituted, and wherein A and C may be attached to the same or different rings;

A is an organic moiety comprising an acid halide, sulfonyl halide, isocyanate or isocyanate equivalent, or activated ester functional group; and

C is an organic moiety comprising an aldehyde or ketone functional group.

Preferred values for A, B, and C are as described above for the first aspect of the invention.

In certain preferred embodiments, B comprises a ring system with known pharmacological activity. In these embodiments, the libraries produced by the method of the invention preferably comprise compounds with similar biological activity to that associated with the B pharmacophore. Non-limiting examples of such pharmacologically active ring systems are shown in FIG. 2.

The libraries of the invention are useful as a source of biologically active compounds. In some preferred embodiments, the library is designed to maximize structural diversity, with there thus being a substantial likelihood that one or more library compounds possesses useful biological activity toward one or more biological targets. In some other preferred embodiments, the library is designed to maximize structural similarity to a compound with known biological activity, with there thus being a substantial likelihood that one or more library compounds possesses similar biological activity toward the target of interest.

The libraries of the invention are useful for screening proteins of known or unknown function. Furthermore, the libraries may be used as tools to probe the three dimensional shapes of binding sites on the proteins. In some preferred embodiments, the protein is an enzyme and the library is used to probe the three dimensional shape of the enzyme active site.

The libraries can be used in any application for which it is useful to screen multiple compounds. For example, the libraries have utility in screening assays for compounds of use in the pharmaceutical or agricultural industries. The libraries can be assayed for the discovery of new drugs, herbicides, pesticides, or antimicrobial agents.

The libraries of the invention may be screened to identify which of the component library compounds possess the biological activity of interest. Preferably, the library is designed so as to facilitate the identification of individual library compounds with the desired biological activity. Methods for coding library compounds in a combinatorial library are known to those of skill in the art. Nonlimiting examples for coding methods include radiofrequency tags, nucleic acid tags, calorimetric tags, spectrometric tags (Raman, IR), polyhalogenated benzene tags, and polymorph tags. In certain preferred embodiments, the library compounds are mass-coded, and are distinguished on the basis of their mass spectra.

The libraries may be screened by any method that is appropriate for the biological target and compatible with the method used to deconvolute the mixture and identify individual active library compounds. The screening assay may be a binding assay, wherein compounds that bind to the target are separated from compounds that do not bind to the target, or it may be a functional assay, wherein the effect of the library on a function of target is measured. The concentration of the library will depend on the sensitivity of the instrument, for example, a mass spectrometer, used to detect hits. Typically, the library is used in sufficient concentration that each compound in the library is present at a concentration from about 1.0 nM to about 0.1 mM.

In certain preferred embodiments, a target biomolecule, such as a protein, is contacted with the library of the invention so that biomolecule-ligand complexes form between the biomolecule and any library compounds that are ligands for the biomolecule. Compounds that do not bind the biomolecule are separated from the biomolecule-ligand complexes. The biomolecule-ligand complexes are then dissociated, the ligands are separated, and the identity of the ligands is determined.

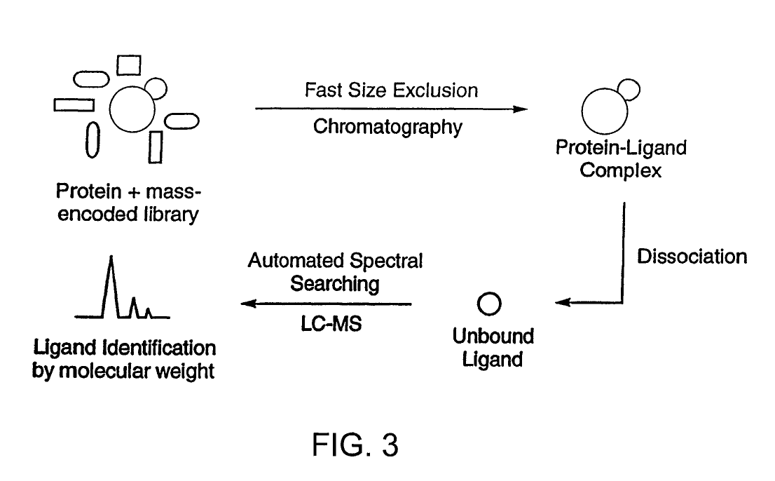

In some preferred embodiments, the library compounds are mass-coded, and are identified by their molecular masses. FIG. 3 is a schematic representation of one such preferred embodiment. Combining a protein and a mass-encoded small molecule library in a physiologically relevant buffer leads to the formation of complexes of the protein with any library compounds that are ligands for the protein. These complexes are preferably separated from non-binding library members by rapid, low temperature (<10 seconds at 0° C.) size exclusion chromatography (SEC). In contrast to other affinity selection methods, such as spin column (Huyer et al., Anal. Biochem. 1998, 258, 19-30) or ultrafiltration (Zhao et al., J. Med. Chem. 1997, 40, 4006-4012), a rapid SEC separation insures that even weakly bound ligands are captured for identification as possible lead structures. The SEC band containing the protein-ligand complexes preferably is immediately analyzed by LC/MS under conditions (e.g., acid, basic, chemical-denaturing) such that ligands dissociate from the protein. In some preferred embodiments, the liquid chromatography step is performed on a reverse-phase chromatography column, which is preferably maintained at 60° C. to promote dissociation of ligands from the complex. The ligand is eluted into a high-resolution mass spectrometer for analysis; automated software algorithms search the mass spectral data to identify ligands by virtue of their molecular weight.

Preferably, a liquid chromatography/mass spectroscopy (LC/MS) analysis of the protein itself (i.e., the protein that has not been exposed to the library) is first performed to provide a background value. The background value is used to eliminate chemical noise, including noise resulting from protein breakdown products, contaminated solvents and buffers, machine contamination, and previous chemicals used in the LC/MS, as well as system electronic noise. The SEC band containing the protein-ligand complexes is then analyzed by LC/MS under conditions (e.g., acid, basic, chemical-denaturing) such that ligands dissociate from the protein. This output is compared to the background at each mass-to-charge (m/z) value corresponding to a library compound. If the expected ligand signal is above the measured background level, a possible hit is recorded. By matching the empirically obtained mass values and calculated mass values, the molecular weights of library molecules that are able to bind to the target protein are deduced. Because this method directly identifies the compound of interest, the incidence of false positives is very low.

Ideally, the structures of the library molecules of interest are deduced directly from their molecular weights. However, if the structure of a library molecule is not uniquely determined by its molecular weight, an iterative process is used to identify the structure of the biologically active library molecule. The iterative process involves generating a second combinatorial library using only those building blocks which the mass spectroscopy data indicate form one of the structures that corresponds to a hit mass value. As a result, the second library contains the possible hit structure in a much smaller combinatorial library, a factor which facilitates mass spectroscopy analysis. The second library is screened and subjected to mass spectroscopy. The empirical mass values are compared to the theoretic mass values (as described above) to deduce the structures of the library molecules of interest. This process is repeated until there is sufficient molecular weight data to determine the structure of the biologically active library molecule.

Certain particularly preferred methods for screening libraries and identifying active library compounds are described in Nash et al., WO 99/35109, and in Birnbaum et al., WO 00/22649, each of which is hereby incorporated by reference in its entirety.

In a third aspect, the invention provides a compound of the formula A-B-C, wherein

B comprises from 1 to about 4 carbocyclic or heterocyclic rings, any of which rings may be optionally substituted, and wherein A and C may be attached to the same or different rings;

A is an organic moiety comprising an acid halide, sulfonyl halide, isocyanate or isocyanate equivalent, or activated ester functional group; and

C is an organic moiety comprising an aldehyde or ketone functional group.

The compounds of the invention are useful as core molecules for the preparation of combinatorial libraries, as described above. The compounds of the invention are also useful as cross-linking reagents. Cross-linking reagents have two or more reactive groups connected by a linker. In the compounds of the invention, A and C comprise reactive groups, and B is a linker connecting the reactive groups. Cross-linking reagents may be used to join two molecules or two remote portions of the same molecule. For example, cross-linking reagents are useful for conjugation or immobilization of proteins and nucleic acids.

Cross-linking reagents are useful in the study of biological macromolecules, by enabling the selective or site-specific chemical cross-linking of one macromolecule to another molecule. The reactive centers of the cross-linking reagent may either be chemically equivalent (homofunctional cross-linking reagents) or chemically different (heterofunctional cross-linking reagents). Homofunctional agents enable the cross-linking of molecules that possess the same nucleophilic moieties, while heterofunctional agents enable the selective cross-linking of molecules that contain different nucleophilic moieties.

To identify biological macromolecules that act by binding non-covalently to each other, researchers have used chemical cross-linking agents to trap the non-covalent interaction as a stable, covalently bound species that is amenable to further study. Researchers have also used cross-linking agents to covalently immobilize biological macromolecules on solid surfaces for applications such as affinity chromatography and immunological assays, including ELISA assays. Researchers have also used cross-linking reagents to covalently attach fluorescent or radiolabeled small molecules to the biological macromolecule for use in diagnostic imaging applications. The use of cross-linking reagents is further described, e.g., by Mattson et al., Mol. Biol. Repts., 17: 167-183 (1993), and by Hermanson, Bioconjugate Techniques, Academic Press: New York, 1996, p. 728.

The term “combinatorial library”, as used herein, refers to a collection of compounds that is synthesized from combinations of two or more starting components. At least some of the compounds must differ from at least some of the other compounds in the library. A library preferably includes at least 100 compounds, and more preferably includes at least 500, 1000, 5000, or more compounds.

The term “library compounds”, as used herein, refer to the molecules that are in a combinatorial library. Library compounds are the products of reactions between cores and building blocks.

The term “deconvolute”, as used herein, refers to a process of determining which compound in a mixture is responsible for an observed activity.

The term “cores”, as used herein, refer to molecules having at least two reactive centers that react with functional groups of building blocks. The terms “core”, “core molecule”, and “core compound” all refer to these molecules, and are used interchangeably. The term “dicore” refers to a core molecule having two reactive centers. The term “tricore” refers to a core molecule having three reactive centers. The term “tetracore” refers to a core molecule having four reactive centers.

A “reactive center”, as used herein, refers to a functional group of a core that is capable of forming a linkage (e.g., a covalent bond) with a complementary functional group of a building block. For example, an electrophilic reactive center, including without limitation an acid halide or activated ester, is capable of forming a linkage with a complementary nucleophilic building block functional group, including without limitation an amine, hydroxy, or thiol functional group.

The term “building blocks”, as used herein, refer to molecules having at least one functional group that can react with the reactive centers of core molecules to form combinatorial library molecules.

A “rotatable bond”, as used herein, refers to a bond about which rotation can occur such that the relative spatial orientation of reactive centers on a core changes or the relative spatial orientation of building block moieties bonded to a core moiety changes.

“Spatial orientation”, as used herein, refers to the placement in space of at least two moieties (attached to the same molecule, such as a core) relative to one another.

The term “organic moiety”, as used herein, refers to a group having from 1 to about 25 carbon atoms; from 0 to about 10 heteroatoms selected from the group consisting of oxygen, nitrogen, and sulfur; and from 0 to about 6 halogen atoms.

A “carbocyclic ring”, “carbocyclic group” or “carbocycle”, as used herein, is a ring structure having from 3 to about 8, preferably 3, 5, or 6 carbon atoms, any of which atoms may be optionally substituted. Some carbocyclic rings are saturated or partially unsaturated. Such saturated or partially unsaturated carbocyclic rings include, without limitation, cyclopropyl, cyclopentyl, cylohexyl, cylopentenyl, cyclohexenyl, and cyclohexadienyl. Some other carbocyclic rings are aromatic rings.

A “heterocyclic ring”, “heterocylic group”, or “heterocycle” is a ring structure having from 3 to about 8 atoms, wherein one or more atoms are selected from the group consisting of N, O, and S. The heterocyclic ring may be optionally substituted on carbon at one or more positions. The heterocyclic group may also independently be substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Some heterocylic rings are saturated or partially unsaturated. Examples of saturated heterocyclic rings include, without limitation, epoxide, aziridine, tetrahydrofuran, pyrrolidine, piperidine, piperazine, thiazolidine, oxazolidine, oxazolidinone, and morpholine. Some other heterocyclic rings are aromatic rings.

An “aromatic ring” or “aryl group”, as used herein, comprises 5 or 6 ring atoms, and has 6 π electrons shared in a cyclic array. The terms “aromatic ring” and “aryl group” are intended to include heteroaryl groups, which have, in addition to carbon atoms, from one to about four, preferably one or two, heteroatoms selected from the group consisting of N, O, and S. Examples of aromatic rings include, without limitation, benzene, pyridine, pyrimidine, pyrazine, thiophene, furan, pyrrole, imidazole, pyrazole, oxazole, isoxazole, thiazole, triazole, and tetrazole.

The aromatic ring may be fused to one or more other aromatic rings to form a fused aromatic ring system, comprising 5 to 14 ring atoms, preferably 5, 6, 9, or 10 ring atoms, and having 6, 10, or 14 π electrons shared in a cyclic array. The aromatic ring may also be fused to one or more non-aromatic rings. Examples of fused ring systems include, without limitation, benzofuran, benzothiophene, quinoline, isoquinoline, quinoxaline, tetrahydroquinoline, dibenzofuran.

The term “activated ester group”, as used herein, refers to a —C(O)OR group, wherein OR is a good leaving group. A good leaving group is an organic moiety that can readily stabilize a negative charge and facilitate nucleophilic substitution at a carbonyl carbon. The factors relating to leaving group ability are well-understood by those of ordinary skill in the art, and are found generally described in March, Advanced Organic Chemistry, 2^{nd}Edition (New York: McGraw Hill, 1977), pages 325-331. Preferably, R contains at least one electron withdrawing group. Electron withdrawing groups are well-known to those of skill in the art and include, without limitation carbonyl, chloro, fluoro, and nitro. More preferably, R is haloalkyl, or aryl substituted with at least one electron withdrawing substituent. Alternatively, OR may be a radical formed by deprotonation of an N-hydroxyimide or N-hydroxyheterocycle, including, without limitation, N-hydroxysuccinimide, N-hydroxyphthalimide, and N-hydroxybenzotriazole.

An “isocyanate equivalent”, as used herein is a —NH—C(O)OR moiety, wherein OR is a good leaving group, as described above.

As employed herein, a “substituted” alkyl, aryl, heteroaryl, carbocyclic or heterocyclic group is one having from one to about four, preferably from one to about three, more preferably one or two, non-hydrogen substituents. Suitable substituents include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, thiol, alkanesulfonyl, arenesulfonyl, alkanesulfonamido, arenesulfonamido, aralkylsulfonamido, acyl, acyloxy, cyano, oximino, and ureido groups.

The term “halogen” or “halo” as employed herein refers to chlorine, bromine, fluorine, or iodine.

As herein employed, the term “acyl” refers to an alkylcarbonyl or arylcarbonyl substituent.

The term “acylamino” refers to an amide group attached at the nitrogen atom. The term “carbamoyl” refers to an amide group attached at the carbonyl carbon atom. The nitrogen atom of an acylamino or carbamoyl substituent may be additionally substituted. The term “sulfonamido” refers to a sulfonamide substituent attached by either the sulfur or the nitrogen atom. Unless otherwise explicitly limited, the term “amino” is meant to include NH₂, alkylamino, dialkylamino, arylamino, aralkylamino, and cyclic amino groups.

As used herein, the term “amine” refers to a compound having at least one amino group. Amine building blocks preferably have primary or secondary amino groups, i.e., the amino group is preferably NH₂ or alkylamino.

The term “oximino” refers to a ═N(OH) or ═N(OR) group, wherein R is alkyl, aryl, aralkyl, sulfonyl, or acyl. Unless otherwise explicitly limited, the term “oximino” is meant to include oximes of either E- or Z-configuration, or mixtures thereof.

The term “ureido” as employed herein refers to a substituted or unsubstituted urea moiety.

The term “pharmacophore”, as used herein, refers to a chemical moiety that is associated with a particular pharmacological activity.

The present invention will now be illustrated by the following examples, which are not intended to be limiting in any way.

### EXAMPLES

### Example 1

### General Procedure for Activation of Aldehyde (or Ketone) Carboxylic Acids

To a solution or suspension of the aldehyde/carboxylic acid (19.9 mmol) in dichloromethane (DCM) (240 mL) was added a solution of pentafluorophenol (22 mmol) and dicyclohexylcarbodiimide (DCC, 22 mL of 1 M solution in DCM). The reaction was stirred at room temperature until the reaction was complete (1-4 h), indicated by the presence of a less polar compound and near complete consumption of starting material (TLC, with DCM or DCM/EtOAc solvents). The reactions were then cooled in an ice bath for 20 min before they were filtered and the insoluble urea of DCC (DCU) discarded. The filtrate was then concentrated and purified using silica gel chromatography with DCM or DCM/EtOAc as the eluant(s). The yields ranged from 50 to 95%.

### Example 2

### Synthesis of Core 1

4-Formyl benzoic acid was purchased from Aldrich Chemical Company and reacted using the above protocol to give a white solid in 94% yield.

¹H NMR (300 MHz, CDCl₃): δ10.17 (s, 1 H), 8.37 (d, J=9.7 Hz, 2 h), 8.06 (d, J=9.6 Hz, 2 H).

### Example 3

### Synthesis of Core 2

3-Formyl benzoic acid was purchased from Aldrich Chemical Company and reacted using the above protocol to give a white solid in 95% yield.

¹H NMR (300 MHz, CDCl₃): δ10.14 (s, 1 H), 8.70 (s, 1 H), 8.45 (dd, J=7.9, 1.4 Hz, 1 H), 8.23 (dd, J=7.8,1.3 Hz, 1 H), 7.76 (t, J=7.8, 1 H).

### Example 4

### Synthesis of Core 3

4-Formylphenoxyacetic acid was purchased from Lancaster Chemical Company and reacted using the above protocol to give a white solid in 60% yield.

¹H NMR (300 MHz, CDCl₃): δ6 9.91 (s, 1 H), 7.88 (d, J=6 Hz, 2 H), 7.06 (d, J=6 Hz, 2 H), 5.07(s,2H).

### Example 5

### Synthesis of Core 4

2-Formylphenoxyacetic acid was purchased from Aldrich Chemical Company and reacted using the above protocol to give a white solid in 56% yield.

¹H NMR (300 MHz, CDCl₃): δ10.55 (s, 1 H), 7.89 (dd, J=4.5, 1 Hz, 1 H), 7.58 (td (J=5,1 Hz, 1 H), 7.14 (t, J=4.8 Hz, 1 H), 6.92 (d, J=4.8 Hz, 1 H), 5.12 (s, 2 H).

### Example 6

### Synthesis of Core 5

5-Formyl-2-furancarboxylic acid was purchased from TCI Chemical Company and reacted using the above protocol to give a white solid in 70% yield.

¹H NMR (300 MHz, CDCl₃): δ9.87 (s, 1 H), 7.56 (d, 2.7 Hz, 1 H), 7.35 (d, J=2.7 Hz, 1 H).

### Example 7

### Synthesis of Core 6

Levulinic acid was purchased from Aldrich Chemical Company and reacted using the above protocol to give an oil in 50% yield.

¹H NMR (300 MHz, CDCl₃): δ2.91 (m, 4 H), 2.23 (s, 3 H).

### Example 8

### Synthesis of Core 7

3-Hydroxy-4-methoxy benzaldehyde (3.0 g, 19.6 mmol), methyl 4-bromobenzoate (3.51 g, 16.4 mmol) was dissolved in pyridine (33 mL) and the flask was evacuated and back-filled with argon. Dried, powdered potassium carbonate (4.5 g, 32.6 mmol) and copper (I) oxide (2.28 g, 28.5 mmol) were added and the reaction was heated to reflux under argon for 24 h. The reaction was then concentrated, diluted with ethyl acetate (350 mL), and filtered through Celite. The filtrate was then rinsed with 1 N HCl, then 0.1 N NaOH. The organic layer was then concentrated (4.8 g) and chromatographed using 1% acetone in DCM to give a white solid (1.78 g, 38%).

¹H NMR (300 MHz, CDCl₃):δ9.87 (s, 1 H), 8.00 (d, J=8.9 Hz, 2 H), 7.67 (dd, J=8.5, 1.9 Hz, 1 H), 7.58 (d, J=1.9 Hz, 1 H), 7.14 (d, J=8.4 Hz, 1 H), 6.93 (d, J=8.8 Hz, 2 H), 3.90 (s, 3 ), 3.89 (s, 3 H).

The methyl ester/aldehyde diphenyl ether (1.61 g, 5.6 mmol) was taken up in MeOH (17 mL), KOH (330 mg, 5.9 mmol) was added and the reaction was warmed to reflux. After 3 h the reaction was cooled and the reaction was concentrated. Water and ethyl acetate were then added until the mixture dissolved. The solution was acidified to a pH of 2 and the solids removed by filtration (880 mg, 58%).

¹H NMR (300 MHz, CDCl₃): δ9.87 (s, 1 H), 8.01 (d, J=9 Hz, 2 H), 7.77 (d, J=9 Hz, 1 H), 7.13 (d, J=8 Hz), 6.93 (d,J=8 Hz, 2 H), 3.90 (s, 3 H)

The carboxylic acid/aldehyde from above was activated to give its PFP ester/aldehyde derivative in 87% yield using the standard procedure.

¹H NMR (300 MHz, CDCl₃): δ9.90 (s, 1 H), 8.15 (d, J=8.9 Hz, 2 H), 7.80 (dd, J=9, 2 Hz, 1 H), 7.65 (d, 1.9 Hz, 1 H), 7.17 (d, J=8.4 Hz, 1 H), 7.00 (d, J=8.9 Hz, 1 H).

### Example 9

### Standard Procedure for Reaction of Core Molecules with Amines

To distilled THF (9.0 mL) and anhydrous MeOH (400 μL) is added super-dried Amberlite (1.6 g) and amine stock solution (solvent: 10% MeOH/DCM, 0.037 mmol amine, 2.2 equivalents). The core (0.30 mmol total or 0.15 mmol each for two core systems) is then added, and the reaction is stirred for 4 h under inert atmosphere. Borohydride exchange resin (750 mg) is then added, along with anhydrous MeOH (5 mL) The reaction is then stirred for 16 hours at room temperature. DCM (7 mL) is then added, followed by an aldehyde resin (700 mg) to scavenge unreacted amine. The reactions may be filtered after stirring for 4-24 hours.

### Example 10

### Test reactions of Cores 1-5 with amines

Each of Cores 1-5 was combined with each of amine building blocks 2-(2-aminoethyl)pyridine (1), 3-pentylamine (2), tyramine (3), and (S)-methioninol (4) according to the following procedures. Stock solutions of the building blocks were prepared by dissolving 3.24 mmol of the building block in 4 mL DCM. Aliquots of 330-340 μL were used for each reaction.

To distilled THF (4.0 mL) and anhydrous MeOH (500 μL) is added super-dried Amberlite (650 mg) and an amine building block (0.27 mmol, 2.2 equiv.). The core (0.12 mmol) is then added. The reaction is stirred for 4 hours at room temperature. Methanol (anhydrous, 2.5 mL) is added, along with borohydride resin (300 mg). The reaction is then stirred for 16 hours at room temperature. DCM (4 mL) is then added, followed by aldehyde resin (200 mg). The reactions were checked by TLC, using DCM:MeOH:TEA (95:5:5) as the elution solvent. LC and MS samples were prepared as 1 mg/mL solutions for HPLC and 10 μg/mL solutions for MS.

The yields of the reactions are shown in the following table.

| Core | Building Block | Crude Yield |
|---|---|---|
| 1 | 1 | 77% |
| 1 | 2 | 77% |
| 1 | 3 | 70% |
| 1 | 4 | 70% |
| 2 | 1 | 86% |
| 2 | 2 | 74% |
| 2 | 3 | 77% |
| 2 | 4 | 63% |
| 3 | 1 | 77% |
| 3 | 2 | 74% |
| 3 | 3 | 80% |
| 3 | 4 | 68% |
| 4 | 1 | 70% |
| 4 | 2 | 79% |
| 4 | 3 | 68% |
| 4 | 4 | 70% |
| 5 | 1 | 79% |
| 5 | 2 | 74% |
| 5 | 3 | 83% |
| 5 | 4 | 71% |

This test reaction protocol was followed for additional amine building blocks. All reactions in which a single peak (not matching the retention time of the starting epoxide or amine) accounted for >80% of the HPLC area above noise level, and whose mass spectrum matched that predicted for the desired amine were deemed to have “passed” this quality control (QC) protocol.

After confirming that individual amines would each react to form a pure compound, other experiments were conducted to show that the amines were competitive in reactivity. The experiments were run with representative sets of two to five amines, and the mass spectra were evaluated to determine whether mass spectra of the product mixtures showed distinct masses corresponding to the desired compounds.

### Example 11

### Addition of benzylamine to Core 1

To super-dried Amberlite resin (0.75 g) was added THF (distilled, 2 mL) and benzylamine (33 μL, 0.3 mmol). PFP/aldehyde Core 1 was added as a solid, along with 0.5 mL THF. The reaction mixture was stirred under argon at room temperature for 2 hours. MeOH (anhydrous, 2 mL) was then added, along with borohydride resin (270 mg). The reaction flask was capped with a rubber septum; a 21-gauge needle was placed in the septa as a vent. The reaction was stirred overnight at room temperature. 46 hours after the addition of borohydride resin, the reaction appeared to be almost complete by TLC (95:5:5 DCM:MeOH:TEA); there was only a slight impurity. No significant difference was observed after an additional two hours.

DCM (2 mL) and aldehyde resin (200 mg) were added. The mixture was stirred for 4 hours, and then filtered. The filtrate was concentrated to yield 43 mg.

### Example 12

### Addition of benzylamine to Core 1

To super-dried Amberlite resin (0.75 g) was added THF (distilled, 2 mL) and benzylamine (33 μL, 0.3 mmol). PFP/aldehyde Core 1 was added as a solid, along with 0.5 mL THF. The reaction mixture was stirred under argon at room temperature for 2 hours. MeOH (anhydrous, 2 mL) was added, along with borohydride resin (325 mg). The reaction flask was capped with a rubber septum; a 21-gauge needle was placed in the septa as a vent. The reaction was stirred overnight at room temperature. It was checked by TLC (95:5:5 DCM:MeOH:TEA). 46 hours after the addition of borohydride resin, the reaction appeared to be almost complete by TLC (95:5:5 DCM:MeOH:TEA); there was only a slight impurity. No significant difference was observed after an additional two hours.

Additional borohydride resin (163 mg) was added. Two hours after the addition of borohydride resin, the reaction was checked by TLC. The reaction did not appear to have progressed any further. DCM (2 mL) and aldehyde resin (200 mg) were added. After stirring for 4 hours, the reaction was filtered, and the filtrate was concentrated. The yield was 41 mg.

### Example 13

### Reaction of Core 1 with a Mixture of Benzylamine and Cyclohexylamine

To super-dried Amberlite resin (0.75 g) was added THF (distilled, 2 mL) and benzylamine (33 μL, 0.3 mmol) and cyclohexylamine (34.3 μL, 0.3 mmol). Core 1 was added as a solid, along with 0.5 mL THF. The reaction mixture was stirred under argon at room temperature for 2 hours. MeOH (anhydrous, 2 mL) was added, along with borohydride resin (650 mg). The reaction flask was capped with a rubber septum; a 21-gauge needle was placed in the septa as a vent. The reaction was stirred overnight at room temperature. It was checked by TLC (95:5:5 DCM:MeOH:TEA). 46 hours after the addition of borohydride resin, the reaction be almost complete by TLC (95:5:5 DCM:MeOH:TEA); there was only a slight impurity. No significant difference was observed after an additional two hours.

DCM (4 mL) and aldehyde resin (400 mg) were added. The mixture was stirred 4 hours, and then filtered. The filtrate was concentrated to yield 525 mg.

### Example 14

### Addition of 2-Methoxybenzylamine Amine to 7-Methoxy-2-Tetralone

To MeOH (1.5 mL) was added the amine (45 mg, 0.33 mmol), then the tetralone (44 mg, 0.25 mmol). The reaction mixture was stirred for 4 hours at room temperature. Borohydride resin (220 mg) was added and the resulting mixture was stirred overnight at room temperature. The reaction was monitored by TLC (eluting with 95:5:5 DCM:MeOH:TEA) and the reaction showed one major spot which was more polar than the starting tetralone after 16 h Aldehyde resin (260 mg) was added along with DCM (2 mL). The mixture was filtered and concentrated. Evaporation gave a tan oil whose mass spectra matched the desired compound and HPLC showed to be >85% pure. This example demonstrates that amines can be added to cyclic ketones, and the minimum ions thus formed can be reduced to form amines under conditions similar to the aldehyde/active ester examples.

### Example 15

### Addition of 2-Methoxybenzylamine to 2-Methoxyphenylacetone

To MeOH (1.5 mL) was added the amine (45 mg, 0.33 mmol), and then the phenylacetone (39 μL, 0.25 mmol). The reaction mixture was stirred for 4 hours at room temperature. Borohydride resin (220 mg) was added and the resulting mixture was stirred overnight at room temperature. The reaction was monitored by TLC (eluting with 95:5:5 DCM:MeOH:TEA) and was complete after 16 h. Aldehyde resin (260 mg) was added along with DCM (2 mL). The mixture was then filtered and concentrated. The sample was yellowish in color and gave one major spot that was more polar than the starting phenylacetone. Evaporation gave a yellow oil whose mass spectra matched the desired compound and HPLC showed to be >85% pure. This example demonstrates that amines can be added to acyclic ketones, and the iminium ions thus formed can be reduced to form amines under conditions similar to the aldehyde/active ester examples.

### Example 16

### Addition of butylamine to Core 6

To distilled THF (5 mL) was added super-dried Amberlite (1.3 g) and the amine (36 μL, 0.36 mmol, 2.4 equivalents). Core 6 (43 mg, 0.15 mmol) was added. The reaction mixture was allowed to stir 4 hours at room temperature. MeOH (3 mL anhydrous) was added along with borohydride resin (400 mg). The resulting mixture was stirred overnight. 5 mL of DCM and 600 mg of aldehyde resin were added, and the reaction mixture was allowed to stir for 4 days. It was then filtered, and the filtrate was concentrated. The crude yield of the reaction was 36 mg (100%); the desired compound was the major component as characterized by HPLC, NMR, and MS. This example demonstrates that amines can be added to a core having a ketone and an activated ester group. The addition can be followed by reduction using a borohydride resin to produce a compound having an amine group and an amide group.

### Example 17

### Addition of tyramine to Core 6

To distilled THF (5 mL) was added super-dried Amberlite (1.3 g) and the amine (49.8 mg, 0.36 mmol, 2.4 equivalents). Core 6 (43 mg, 0.15 mmol) was added, and upon addition of the core, tyramine went into solution. The reaction mixture was allowed to stir 4 hours at room temperature. MeOH (3 mL, anhydrous) was added along with borohydride resin (400 mg). The resulting mixture was stirred overnight. 5 mL of DCM and 600 mg of aldehyde resin were added and the mixture was allowed to stir 4 days. It was then filtered, and the filtrate was concentrated. The crude yield of the reaction was 42 mg (78%); the desired compound was the major component as characterized by HPLC, NMR, and MS.

### Example 18

### Preparation of combinatorial libraries using Cores 1-7

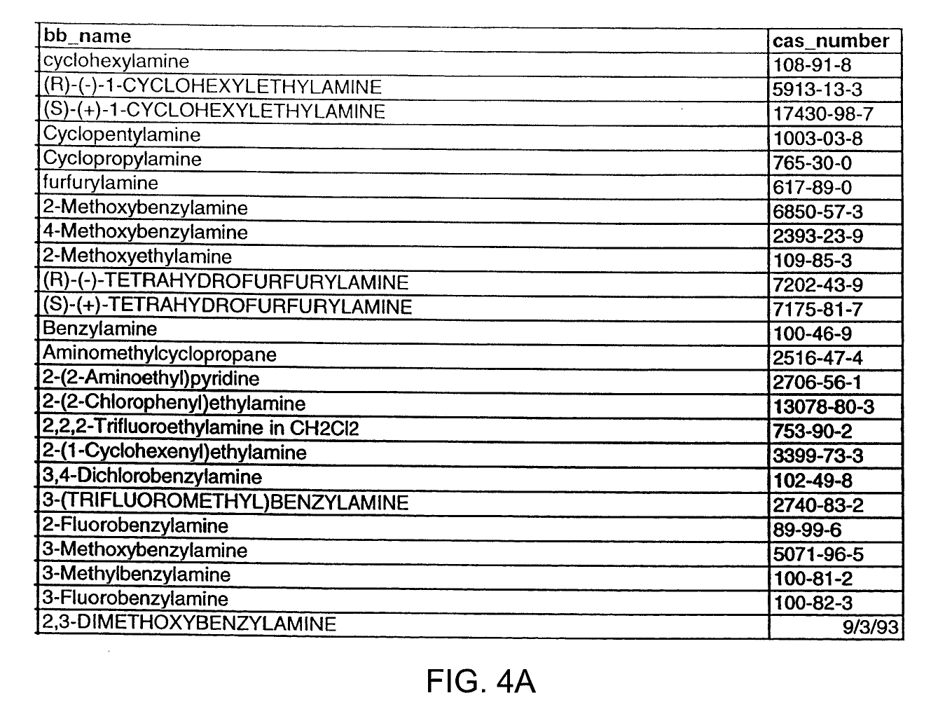

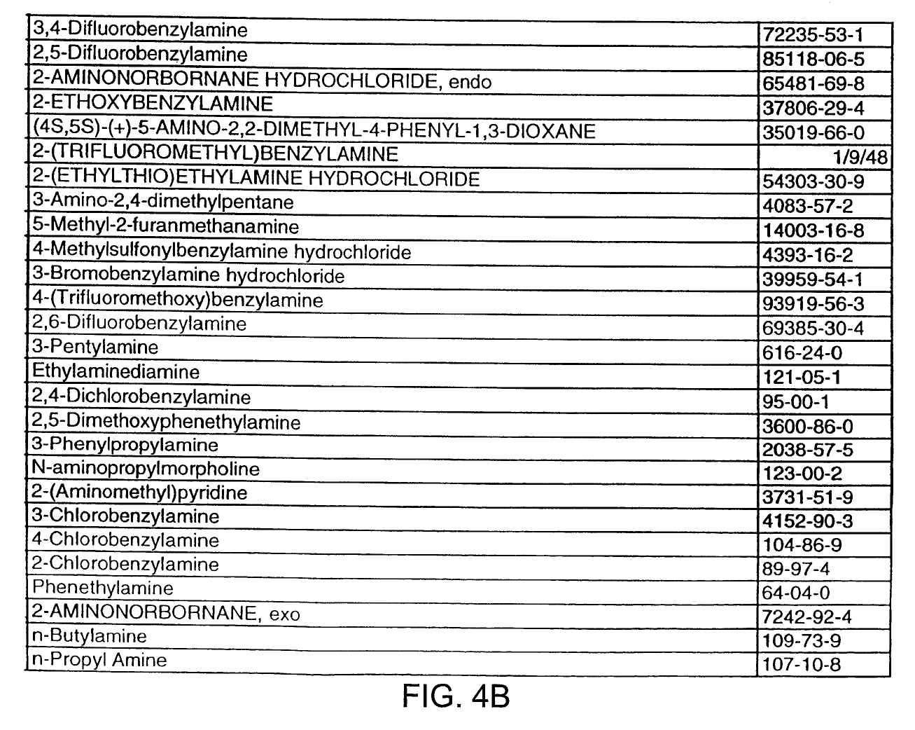

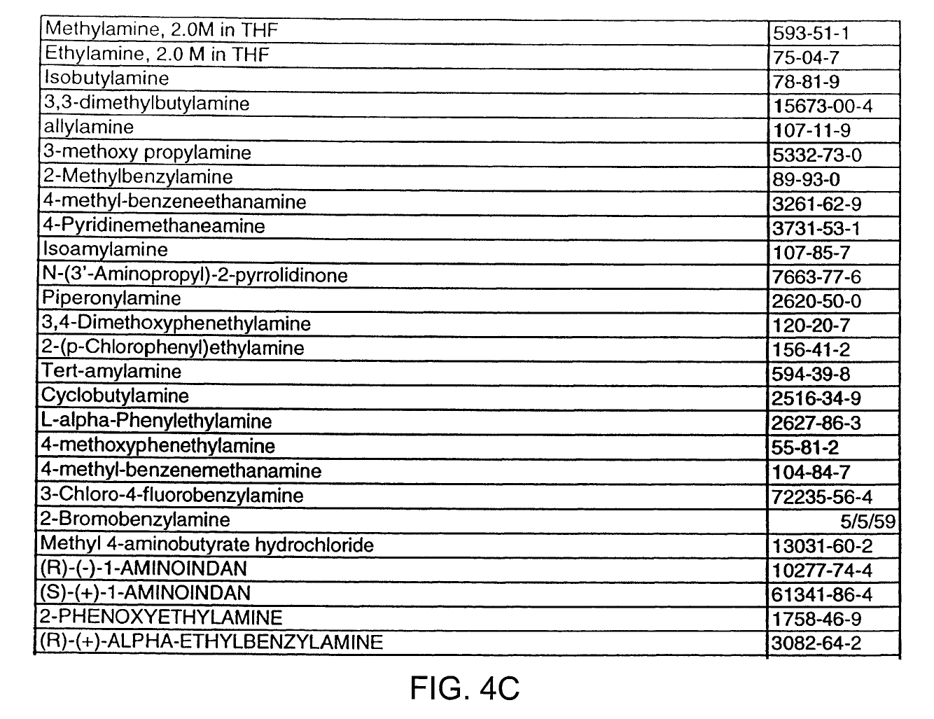

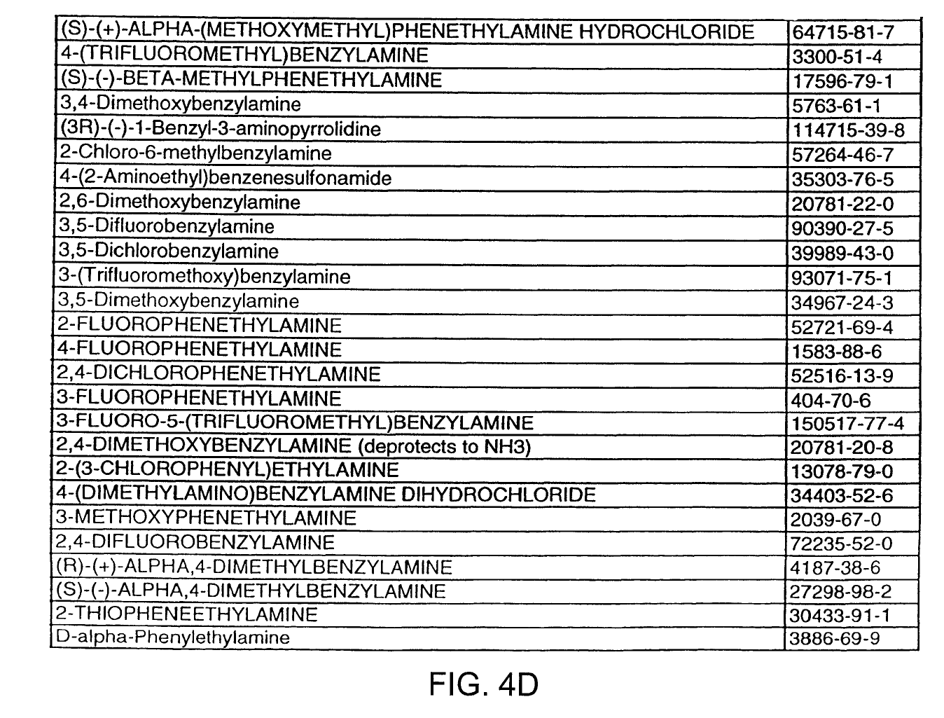

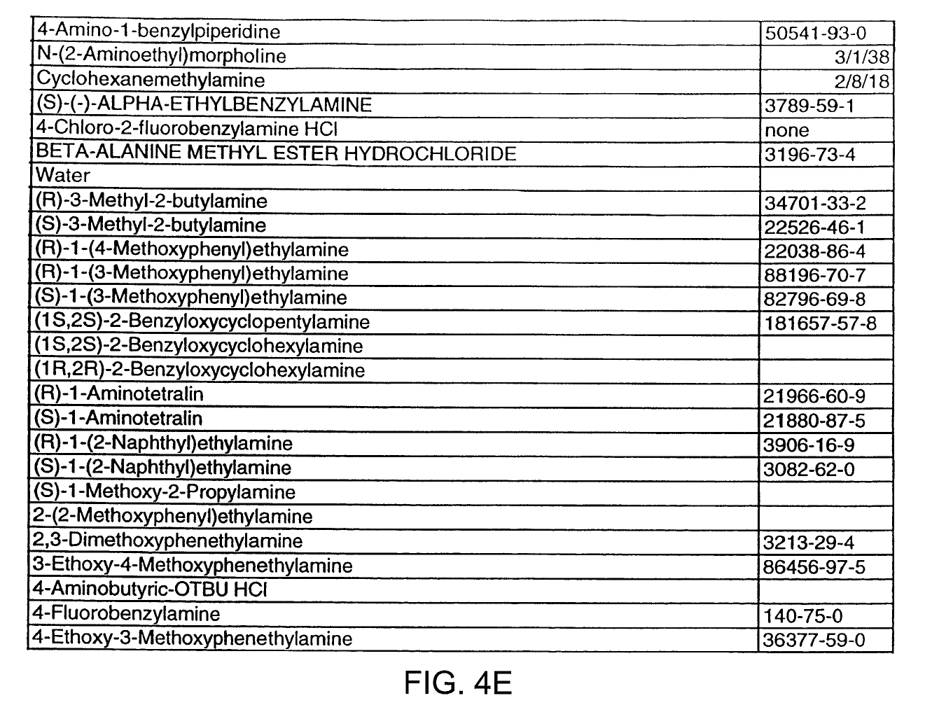

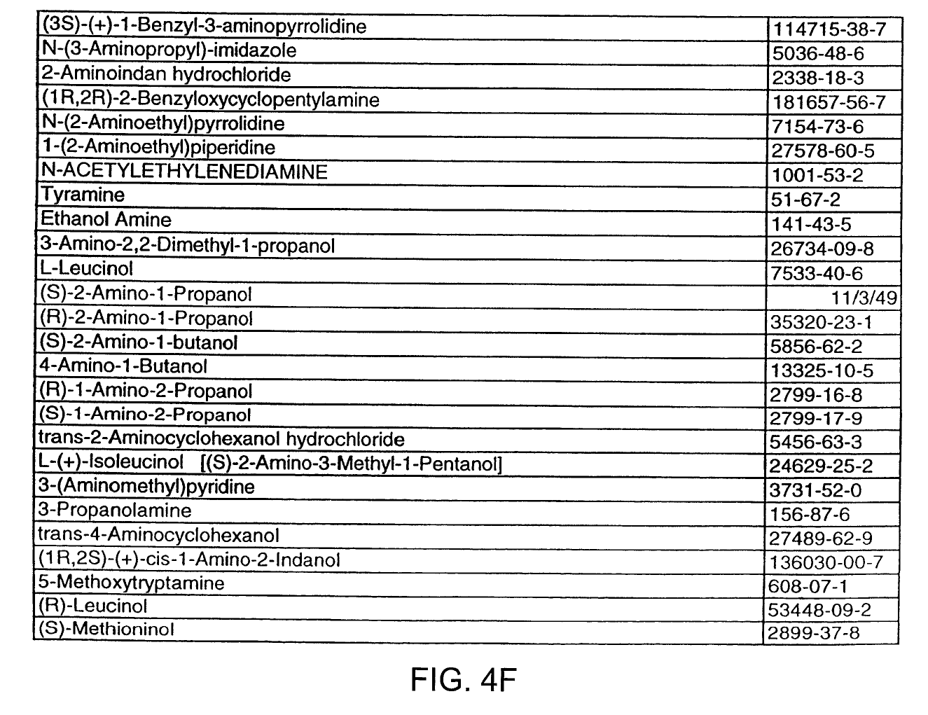

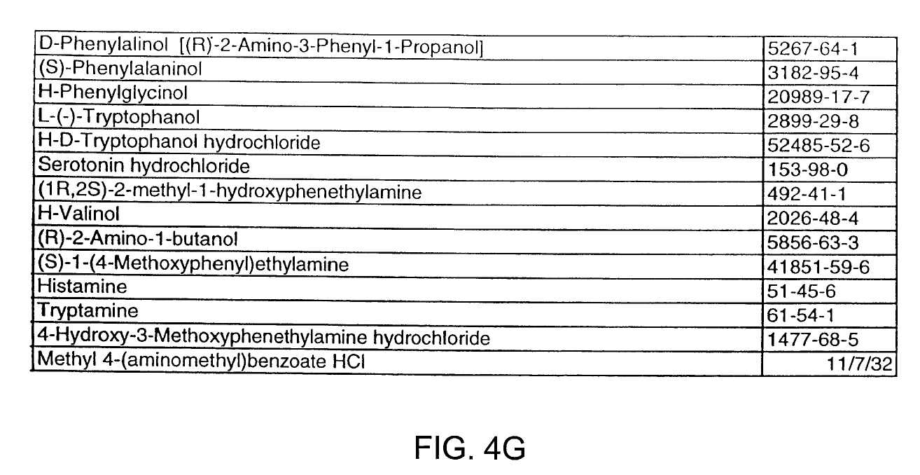

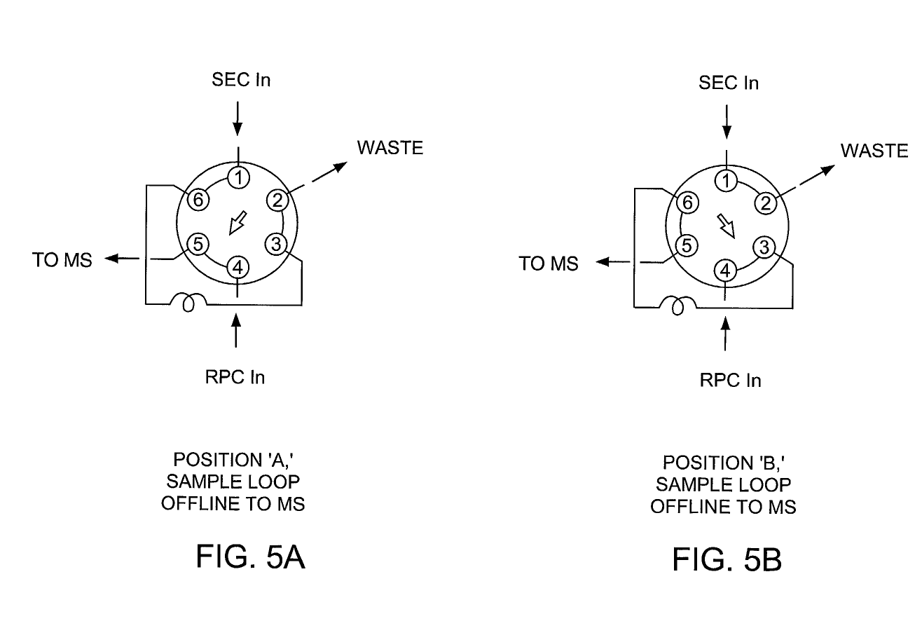

Combinatorial libraries were prepared using cores 1-7 and building blocks selected from those shown in FIG. 4 as follows:

Amine building blocks sets were selected according to the procedures described in Example 10. Each set was selected to form a mass coded library with 4% redundancy at 0.01 AMU.

Stock solutions of the building blocks were prepared as follows. All building blocks in a given set were taken up in 10% MeOH in DCM, 220 mmol of each building block plus 40 mL solvent. The stock solution was used as 5-mL aliquots per library flask (0.037 mmol).

To each of 16 reaction tubes was added 0.9 mL distilled THF, 400 μL anhydrous MeOH, 1.6 g super-dried Amberlite resin, and 5 mL of a building block stock solution. Stock solutions of each of the four sets of building blocks A-D were added to each of four tubes. The tubes were purged with argon and kept under argon at positive pressure. To each tube was added one core; each of the four cores was added a tube containing each building block set. The 16 mixtures were stirred for 4 hours. 5 mL of anhydrous MeOH and 750 mg of borohydride resin were then added. The mixtures were stirred for 16 hours. 700 mg of aldehyde resin was added. The library mixtures were stirred for 16 hours, after which time they were filtered and dried down in a Savant speedvac. The dried libraries were taken up in 23 mL 3:1 DCM:MeOH and aliquoted into 45 vials (500 μL) each. Final evaporation in the vials was done by Savant speedvac to give ˜3 mg per vial.

While the foregoing invention has been described in some detail for purposes of clarity and understanding, it will be appreciated by one skilled in the art from a reading of this disclosure that various changes in form and detail can be made without departing from the true scope of the invention and appended claims.

What is claimed is:

1. A method of forming a combinatorial library of compounds, the method comprising reacting a plurality of core molecules with a mixture of nucleophilic building blocks in a reaction vessel to form a library of compounds, wherein each of said core molecules comprises (i) an acid halide, sulfonyl halide, isocyanate or isocyanate equivalent, or activated ester functional group; and (ii) an aldehyde or ketone functional group.

2. The method of claim 1, wherein the mixture of nucleophilic building blocks comprises at least one amine.

3. The method of claim 1, wherein substantially all of the nucleophilic building blocks are amines.

4. The method of claim 1, wherein substantially all of the core molecules are the same.

5. The method of claim 1, wherein the plurality of core molecules comprises at least two different core molecules.

6. The method of claim 3, wherein the set of nucleophilic building blocks that are each contacted with and can each react with the acid halide, sulfonyl halide, isocyanate or isocyanate equivalent, or activated ester reactive centers are different from the set of nucleophilic building blocks that are each contacted with and can each react with the aldehyde or ketone reactive centers.

7. The method of claim 4, wherein the core molecule comprises two acid halide, sulfonyl halide, isocyanate or isocyanate equivalent, or activated ester functional groups.

8. The method of claim 4, wherein the core molecule comprises three acid halide, sulfonyl halide, isocyanate or isocyanate equivalent, or activated ester functional groups.

9. The method of claim 1, wherein each of said core molecules comprises an activated ester group.

10. The method of claim 9, wherein the activated ester group is a pentafluorophenyl ester group.

11. The method of claim 9, wherein the activated ester group is a dinitrophenyl ester group.

12. The method of claim 1, wherein the aldehyde or ketone functional group is a terminal aldehyde or ketone.

13. The method of claim 1, wherein said core molecule has the formula A-B-C, wherein
B comprises from 1 to about 4 carbocyclic or heterocyclic rings, any of which rings may be optionally substituted, and wherein A and C may be attached to the same or different rings;
A is an organic moiety comprising an acid halide, sulfonyl halide, isocyanate or isocyanate equivalent, or activated ester functional group; and
C is an organic moiety comprising an aldehyde or ketone functional group.

14. The method of claim 13, wherein at least one of the rings is an aromatic ring.

15. The method of claim 13, wherein B comprises a fused bicyclic or tricyclic ring system.

16. The method of claim 13, wherein B comprises two rings connected by a covalent bond.

17. The method of claim 13, wherein A and C are attached to the same ring.

18. The method of claim 14, wherein the ring to which A and C are attached is a benzene ring.

19. The method of claim 13, wherein
A has the formula —Y¹—W, where:
W is an isocyanate or isocyanate equivalent, acid halide, or sulfonyl halide functional group, or W has the formula —C(O)—OR¹, where R¹ is selected from the group consisting of haloalkyl and aryl substituted with at least one electron withdrawing substituent, or —OR¹ is a radical formed by deprotonation of an N-hydroxyheterocycle or N-hydroxyimide;
Y¹ is absent or comprises a linking chain of from 1 to about 6 contiguous atoms independently selected from the group consisting of carbon, nitrogen, oxygen, or sulfur, wherein the carbon and nitrogen atoms may be optionally substituted and the nitrogen and sulfur atoms may be optionally oxidized, and wherein any of the contiguous atoms of the chemical linkage may form part of a ring structure; and
C has the formula —Y²—Z, where Y² is as defined above for Y¹ and Z is an aldehyde or ketone.

20. The method of claim 19, wherein R¹ is selected from the group consisting of succinimide, phthalimide, perfluoroalkyl, pentafluorophenyl, dinitrophenyl, nitrophenyl, difluorophenyl, fluorophenyl, trifluorophenyl, chlorophenyl, dichlorophenyl, chloronitrophenyl, and tetrafluoronitrophenyl.

21. The method of claim 19, wherein W is selected from the group consisting of acid halide, sulfonyl halide, dinitrophenyl ester, and pentafluorophenyl ester.

22. The method of claim 19, wherein Y¹ comprises a W group.

23. The method of claim 19, wherein Y² comprises a W group.

24. The method of claim 19, wherein the Y¹ or Y² linking chain comprises an ester, amide or sulfonamide linkage.

25. The method of claim 19, wherein the Y¹ or Y² linking chain comprises an ether linkage.

26. The method of claim 19, wherein the Y² linking chain comprises a ring and the aldehyde or ketone functional group is attached to the ring.

27. The method of claim 1, wherein said mixture of amines comprises primary amines.

28. The method of claim 1, wherein at least 90% of said library compounds each comprise a secondary amine functional group and an amide, sulfonamide, urea, ester, carbamate, or sulfonate ester functional group.

29. The method of claim 28, wherein at least 95% of said library compounds each comprise a secondary amine functional group and an amide, sulfonamide, urea, ester, carbamate, or sulfonate ester functional group.

30. The method of claim 29, wherein at least 99% of said library compounds each comprise a secondary amine functional group and an amide, sulfonamide, urea, ester, carbamate, or sulfonate ester functional group.

31. A method of forming a combinatorial library of compounds, the method comprising reacting a plurality of core molecules with a mixture of nucleophilic building blocks in a reaction vessel to form a library of compounds, wherein substantially all of the core molecules have the formula

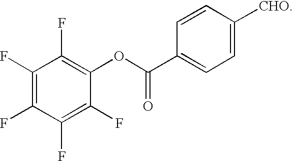

32. A method of forming a combinatorial library of compounds, the method comprising reacting a plurality of core molecules with a mixture of nucleophilic building blocks in a reaction vessel to form a library of compounds, wherein substantially all of the core molecules have the formula

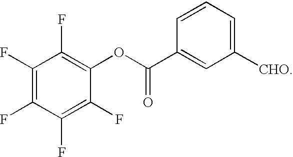

33. A method of forming a combinatorial library of compounds, the method comprising reacting a plurality of core molecules with a mixture of nucleophilic building blocks in a reaction vessel to form a library of compounds, wherein substantially all of the core molecules have the formula

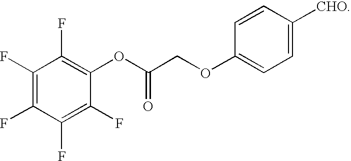

34. A method of forming a combinatorial library of compounds, the method comprising reacting a plurality of core molecules with a mixture of nucleophilic building blocks in a reaction vessel to form a library of compounds, wherein substantially all of the core molecules have the formula

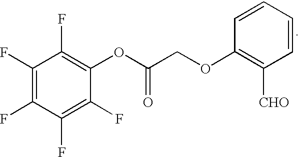

35. A method of forming a combinatorial library of compounds, the method comprising reacting a plurality of core molecules with a mixture of nucleophilic building blocks in a reaction vessel to form a library of compounds, wherein substantially all of the core molecules have the formula

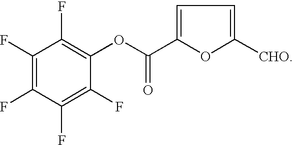

36. A method of forming a combinatorial library of compounds, the method comprising reacting a plurality of core molecules with a mixture of nucleophilic building blocks in a reaction vessel to form a library of compounds, wherein substantially all of the core molecules have the formula

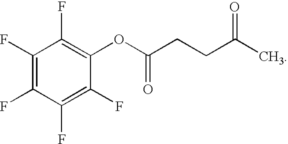

37. A method of forming a combinatorial library of compounds, the method comprising reacting a plurality of core molecules with a mixture of nucleophilic building blocks in a reaction vessel to form a library of compounds, wherein substantially all of the core molecules have the formula

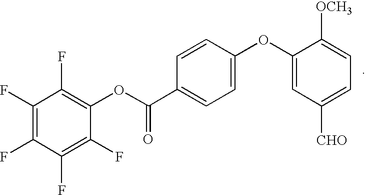

38. A combinatorial library of compounds, wherein said compounds are produced by reacting a plurality of core molecules with a mixture of nucleophilic building blocks, wherein the core molecule comprises (i) an acid halide, sulfonyl halide, isocyanate or isocyanate equivalent, or activated ester functional group; and (ii) an aldehyde or ketone functional group.

39. The library of claim 38, wherein said reacting step comprises sequentially
(i) contacting the core molecules with a mixture of building blocks so that reaction with the acid halide, sulfonyl halide, isocyanate or isocyanate equivalent, or activated ester functional groups is achieved and reaction with aldehyde or ketone functional groups is also achieved to form a mixture of intermediate compounds comprising imine groups; and
(ii) adding a reducing agent to reduce the imine groups formed in step (i).

40. The library of claim 38, wherein said reacting step comprises sequentially
(i) contacting the core molecules with a mixture of amine building blocks so that reaction with the acid halide, sulfonyl halide, isocyanate or isocyanate equivalent, or activated ester functional groups is achieved to produce a first mixture of intermediate compounds;
(ii) contacting the first mixture of intermediate compounds with a mixture of amine building blocks so that reaction with the aldehyde or ketone functional groups is achieved to form a second mixture of intermediate compounds comprising imine groups; and
(ii) contacting the second mixture of intermediate compounds with a reducing agent to reduce the imine groups formed in step (i).

41. A compound having the formula A-B-C, wherein
B comprises from 1 to about 4 carbocyclic or heterocyclic rings, any of which rings may be optionally substituted, and wherein A and C may be attached to the same or different rings;
A is an organic moiety comprising an acid halide, sulfonyl halide, isocyanate or isocyanate equivalent, or activated ester functional group; and
C is an organic moiety comprising an aldehyde or ketone functional group.
